(19) United States
(12) Patent Application Publication
(10) Pub. No.: US 2020/0201434 A1
(43) Pub. Date: Jun. 25, 2020

(54) BIORESPONSIVE VIRTUAL REALITY SYSTEM AND METHOD OF OPERATING THE SAME

(71) Applicant: Samsung Electronics Co., Ltd., Suwon-si (KR)

(72) Inventor: Alireza Aliamiri, San Jose, CA (US)

(21) Appl. No.: 16/280,457

(22) Filed: Feb. 20, 2019

Related U.S. Application Data
(60) Provisional application No. 62/783,129, filed on Dec. 20, 2018.

(51) Int. Cl. G06F 3/01; G06N 3/08; G16H 50/20; A61B 5/16; A61B 5/0205; A61B 5/0484; A61B 5/00

(52) U.S. Cl. CPC G06F 3/015; G06F 3/011; G06N 3/08; G16H 50/20; G06F 3/0346; A61B 5/0205; A61B 5/04842; A61B 5/6803; A61B 5/165

(57) ABSTRACT

A bioresponsive virtual reality system includes: a head-mounted display including a display device, the head-mounted display being configured to display a three-dimensional virtual reality environment on the display device; a plurality of bioresponsive sensors; and a processor connected to the head-mounted display and the bioresponsive sensors. The processor is configured to: receive signals indicative of a user's arousal and valance levels from the bioresponsive sensors; calibrate a neural network to correlate the user's arousal and valance values to a calculated affective state; calculate the user's affective state based on the signals; and vary the virtual reality environment displayed on the head-mounted display in response to the user's calculated affective state to induce a target affective state.

### CROSS-REFERENCE TO RELATED APPLICATION

This utility patent application claims priority to and the benefit of U.S. Provisional Patent Application Ser. No. 62/783,129, filed Dec. 20, 2018 and entitled “METHOD AND APPARATUS FOR AFFECTIVE APPLICATIONS USING VIRTUAL REALITY AND PHYSIOLOGICAL SIGNALS,” the entire content of which is incorporated herein by reference.

### BACKGROUND

### 1. Field

Aspects of example embodiments of the present disclosure relate to a bioresponsive virtual reality system and a method of operating the same.

### 2. Related Art

Virtual reality systems have recently become popular. A virtual reality system generally includes a display device for displaying a virtual reality environment, a processor for driving the display device, a memory for storing information to be displayed on the display device, and an input device for controlling the user's motion in the virtual reality environment. Because virtual reality systems are often intended to provide an immersive environment to a user, the components of the virtual reality system may be housed in a housing that sits on the user's head and moves with the user, such as a headset, and the input device may be one or more gyroscopes and/or accelerometers in the headset. Such a system is often referred to as a head-mounted display (HMD).

The display device may be configured to provide an immersive effect to a user by presenting content, such as a seemingly three-dimensional virtual reality environment, to the user. For example, the virtual reality system may include one or more lenses arranged between the display device and the user's eyes such that one or more two-dimensional images displayed by the display device appear to the user as a three-dimensional virtual reality environment. As used herein, the term “image” and “images” is intended to encompass both still images and moving images, such as movies, videos, and the like.

One method of presenting a three-dimensional image to a user is by using a stereoscopic display that includes two display devices (or, in some cases, one display device configured to display two different images) and one or more magnifying lenses to compensate for the distance from the display device to the user's eyes.

In some instances, the HMD may include gyroscopes, accelerometers, and/or the like to provide head-tracking functionality. By tracking the user's head movements, a fully immersive environment may be provided to the user, allowing the user to “look around” the virtual reality environment by simply moving his or her head. Alternatively, or in combination with, the gyroscopes and/or accelerometers, a controller (e.g., a handheld controller) may be provided to allow the user to “move” around the virtual reality environment. The controller may also allow the user to interact with (or interact with objects and/or characters in) the virtual reality environment.

### SUMMARY

The present disclosure is directed toward various embodiments of a bioresponsive virtual reality system and a method of operating the same.

According to an embodiment of the present disclosure, a bioresponsive virtual reality system includes: a head-mounted display including a display device, the head-mounted display being configured to display a three-dimensional virtual reality environment on the display device; a plurality of bioresponsive sensors; and a processor connected to the head-mounted display and the bioresponsive sensors. The processor is configured to: receive signals indicative of a user's arousal and valance levels from the bioresponsive sensors; calibrate a neural network to correlate the user's arousal and valance values to a calculated affective state; calculate the user's affective state based on the signals; and vary the virtual reality environment displayed on the head-mounted display in response to the user's calculated affective state to induce a target affective state.

The bioresponsive sensors my include at least one of an electroencephalogram sensor, a galvanic skin response sensor, and/or a heart rate sensor.

The bioresponsive virtual reality system may further include a controller.

The galvanic skin response sensor may be a part of the controller.

The bioresponsive virtual reality system may further include an electrode cap, and electrode cap may include the electroencephalogram sensor.

To calibrate the neural network, the processor may be configured to: display content annotated with an expected affective state; calculate the user's affective state based on the signals; compare the user's calculated affective state with the annotation of the content; and when the user's calculated affective state is different from the annotation of the content, modify the neural network to correlate the signals with the annotation of the content.

To vary the virtual reality environment to induce the target affective state, the processor may be configured to use deep reinforcement learning to determine when to vary the virtual reality environment in response to the user's calculated affective state.

According to an embodiment of the present disclosure, a bioresponsive virtual reality system includes: a processor and a memory connected to the processor; a head-mounted display including a display device, the head-mounted display device being configured to present a three-dimensional virtual reality environment to a user; and a plurality of bioresponsive sensors connected to the processor. The memory stores instructions that, when executed by the processor, cause the processor to: receive signals from the bioresponsive sensors; calibrate an affective state classification network; calculate a user's affective state by using the affective state classification network; and vary the virtual reality environment displayed to the user based on the user's calculated affective state.

The affective state classification network may include a plurality of convolutional neural networks, including one convolutional neural network for each of the bioresponsive signals and a final network combining these networks to achieve multi-modal operation.

The affective state classification network may further include a fully connected cascade neural network, the convolutional neural networks may be configured to output to the fully connected cascade neural network, and the fully connected cascade neural network may be configured to calculate the user's calculated affective state based on the output of the convolutional neural networks.

To calibrate the affective state classification network, the memory may store instructions that, when executed by the processor, cause the processor to: input a baseline model that is based on the general population; display annotated content to the user by using the head-mounted display, the annotation indicating an affective state relating to the annotated content; compare the user's calculated affective state with the affective state of the annotation; and when a difference between the user's calculated affective state and the affective state of the annotation is greater than a value, modify the baseline model to correlate the received signals with the affective state of the annotation.

To vary the virtual reality environment, the memory may store instructions that, when executed by the processor, cause the processor to: compare the user's calculated affective state with a target affective state; and when a difference between the user's calculated affective state and the target affective state is greater than a value, vary the virtual reality environment to move the user toward the target affective state.

To vary the virtual reality environment, the memory may store instructions that, when executed by the processor, cause the processor to use a deep reinforcement learning method to correlate variations of the virtual reality environment with changes in the user's calculated affective state.

The deep reinforcement learning method uses Equation 1 as the value function, and Equation 1 is:

Q^{π}(s, a)=E[rₜ₊₁+yrₜ₊₂+y²rₜ₊₃+ . . . |s, a]

wherein: s is the user's calculated affective state; rₜ is the target affective state; a is the varying of the virtual reality environment; π is the mapping of the user's calculated affective state to the varying of the virtual reality environment; Q is the user's expected resulting affective state; and y is a discount factor.

According to an embodiment of the present disclosure, a method of operating a bioresponsive virtual reality system includes calibrating an affective state classification network; calculating a user's affective state by using the calibrated affective state classification network; and varying a three-dimensional virtual reality environment displayed to the user when the user's calculated affective state is different from a target affective state.

The calculating the user's affective state may include: receiving signals from a plurality of biophysiological sensors; inputting the received signals into a plurality of convolutional neural networks, the convolutional neural networks being configured to classify the signals as indicative of the user's arousal and/or valance levels; and inputting the user's arousal and/or valance levels into a neural network, the neural network being configured to calculate the user's affective state based on the user's arousal and/or valance levels.

The biophysiological sensors may include at least one of an electroencephalogram sensor, a galvanic skin response sensor, and/or a heart rate sensor.

The calibrating of the affective state classification network may include: displaying a three-dimensional virtual reality environment having an annotation to the user, the annotation indicating an affective state relating to the virtual reality environment; comparing the user's calculated affective state with the affective state of the annotation; and when a difference between the user's calculated affective state and the affective state of the annotation is greater than a threshold value, modifying the affective state classification network to correlate the received biophysiological signals with the affective state of the annotation.

The varying of the three-dimensional virtual reality environment may include: receiving the target affective state; comparing the user's calculated affective state with the target affective state; varying the three-dimensional virtual reality environment when a difference between the user's calculated affective state and the target affective state is greater than a threshold value; recalculating the user's affective state after the varying of the three-dimensional virtual reality environment; and comparing the user's recalculated affective state with the target affective state.

A deep-Q neural network may be used to compare the user's calculated affective state with the target affective state.

### BRIEF DESCRIPTION OF THE DRAWINGS

FIG. 1 is a schematic illustration of a bioresponsive virtual reality system including a head-mounted display (HMD) on a user according to an embodiment of the present disclosure;

FIGS. 2A-2C are schematic illustrations of the bioresponsive virtual reality system shown in FIG. 1;

FIG. 2D is a schematic illustration of a bioresponsive virtual reality system according to another embodiment;

FIG. 3 shows outputs of an EEG showing different emotional states of a user;

FIG. 4 is a schematic illustration of aspects of a biofeedback response (“bioresponsive”) virtual reality system according to an embodiment of the present disclosure;

FIG. 5 is a diagram illustrating core emotional affects;

FIG. 6 is a schematic diagram illustrating an affective classification neural network of the bioresponsive virtual reality system shown in FIG. 4;

FIG. 7 is a schematic diagram illustrating a control neural network of the bioresponsive virtual reality system shown in FIG. 4; and

FIG. 8 is a flowchart illustrating a method of calibrating the affective classification neural network according to an embodiment of the present disclosure.

### DETAILED DESCRIPTION

The present disclosure is directed toward various embodiments of a bioresponsive virtual reality system and a method of operating the same. According to embodiments of the present disclosure, a bioresponsive virtual reality system includes a head-mounted display device that provides a user with a three-dimensional virtual reality environment, a controller for interacting with the virtual reality environment, and a plurality of biophysiological sensors for monitoring the user's arousal and/or valance levels. During use, the bioresponsive virtual reality system monitors the output of the biophysiological sensors to calculate the user's affective state and may vary the presented (or displayed) virtual reality environment to move the user into a target affective state.

Hereinafter, example embodiments of the present disclosure will be described, in more detail, with reference to the accompanying drawings. The present disclosure, however, may be embodied in various different forms and should not be construed as being limited to only the embodiments illustrated herein. Rather, these embodiments are provided as examples so that this disclosure will be thorough and complete and will fully convey the aspects and features of the present disclosure to those skilled in the art. Accordingly, processes, elements, and techniques that are not necessary to those having ordinary skill in the art for a complete understanding of the aspects and features of the present disclosure may not be described. Unless otherwise noted, like reference numerals denote like elements throughout the attached drawings and the written description, and thus, descriptions thereof may not be repeated.

It will be understood that, although the terms “first,” “second,” “third,” etc., may be used herein to describe various elements, components, and/or layers, these elements, components, and/or layers should not be limited by these terms. These terms are used to distinguish one element, component, or layer from another element, component, or layer. Thus, a first element, component, or layer described below could be termed a second element, component, or layer without departing from the scope of the present disclosure.

It will be understood that when an element or component is referred to as being “connected to” or “coupled to” another element or component, it may be directly connected or coupled to the other element or component or one or more intervening elements or components may also be present. When an element or component is referred to as being “directly connected to” or “directly coupled to” another element or component, there are no intervening element or component present. For example, when a first element is described as being “coupled” or “connected” to a second element, the first element may be directly coupled or connected to the second element or the first element may be indirectly coupled or connected to the second element via one or more intervening elements.

The terminology used herein is for the purpose of describing particular embodiments and is not intended to be limiting of the present disclosure. As used herein, the singular forms “a” and “an” are intended to include the plural forms as well, unless the context clearly indicates otherwise. It will be further understood that the terms “comprises,” “comprising,” “includes,” and “including,” when used in this specification, specify the presence of the stated features, integers, steps, operations, elements, and/or components but do not preclude the presence or addition of one or more other features, integers, steps, operations, elements, components, and/or groups thereof. That is, the processes, methods, and algorithms described herein are not limited to the operations indicated and may include additional operations or may omit some operations, and the order of the operations may vary according to some embodiments. As used herein, the term “and/or” includes any and all combinations of one or more of the associated listed items.

As used herein, the term “substantially,” “about,” and similar terms are used as terms of approximation and not as terms of degree, and are intended to account for the inherent variations in measured or calculated values that would be recognized by those of ordinary skill in the art. Further, the use of “may” when describing embodiments of the present disclosure refers to “one or more embodiments of the present disclosure.” As used herein, the terms “use,” “using,” and “used” may be considered synonymous with the terms “utilize,” “utilizing,” and “utilized,” respectively. Also, the term “example” is intended to refer to an example or illustration.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

A processor, central processing unit (CPU), graphics processing unit (GPU), and/or any other relevant devices or components according to embodiments of the present disclosure described herein may be implemented utilizing any suitable hardware (e.g., an application-specific integrated circuit), firmware, software, and/or a suitable combination of software, firmware, and hardware. For example, the various components of the processor, CPU, and/or the GPU may be formed on (or realized in) one integrated circuit (IC) chip or on separate IC chips. Further, the various components of the processor, CPU, GPU, and/or the memory may be implemented on a flexible printed circuit film, a tape carrier package (TCP), a printed circuit board (PCB), or formed on the same substrate as the processor, CPU, and/or the GPU. Further, the described actions may be processes or threads, running on one or more processors (e.g., one or more CPUs, GPUs, etc.), in one or more computing devices, executing computer program instructions and interacting with other system components to perform the various functionalities described herein. The computer program instructions may be stored in a memory, which may be implemented in a computing device using a standard memory device, such as, for example, a random access memory (RAM). The computer program instructions may also be stored in other non-transitory computer readable media such as, for example, a CD-ROM, flash drive, HDD, SSD, or the like. Also, a person of skill in the art should recognize that the functionality of various computing devices may be combined or integrated into a single computing device or the functionality of a particular computing device may be distributed across one or more other computing devices without departing from the scope of the exemplary embodiments of the present disclosure.

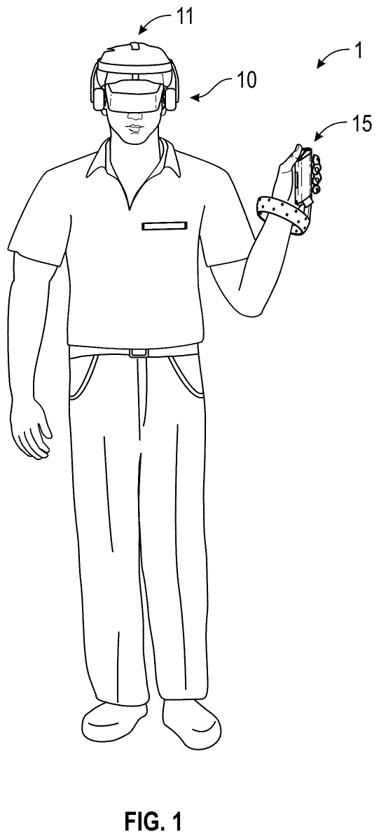

FIG. 1 illustrates a user 1 using a bioresponsive virtual reality system according to an embodiment of the present disclosure. In FIG. 1, the user 1 is illustrated as wearing a head-mounted display (HMD) 10 of the bioresponsive virtual reality system. The HMD 10 may include a housing in which a display device (or a plurality of display devices, such as two display devices) and one or more lenses are housed. The housing may be made of, for example, plastic and/or metal and may have a strap attached thereto to be fitted around the head of user 1.

In some embodiments, the display device may be a smartphone or the like, such that the user 1 may remove the display device from the housing to use the display device independently of the HMD 10 and the bioresponsive virtual reality system and may install the display device into the HMD 10 when he or she wishes to use the bioresponsive virtual reality system. When the HMD 10 includes the removable display device, the display device may include a processor and memory for driving the display device, such as when the display device is a smartphone or the like. In embodiments in which the display device is fixedly mounted to the HMD 10, the HMD 10 may further include a processor and memory separate from the display device. The HMD 10, according to either embodiment, may include a battery pack (e.g., a rechargeable battery pack) to power the display device, processor, and memory. In some embodiments, the HMD 10 may be configured to be connected to an external power supply for long-term uninterrupted viewing. The memory may store thereon instructions that, when executed by the processor, cause the processor to drive the display device to display content, such as images for an immersive virtual reality environment.

The HMD 10 (or the display device when it is a smartphone or the like) may also include one or more gyroscopes, accelerometers, etc. These devices may be used to track the movements of the head of user 1, and the bioresponsive virtual reality system may update the displayed images based on the movement of the user's head.

As described above, the HMD 10 may present (or display) a three-dimensional image (e.g., a virtual reality environment) to the user 1 by using, for example, stereo imaging (also referred to as stereoscopy). Stereo imaging provides the user 1 with an image having three-dimensional depth by presenting two slightly different images to the user's eyes. For example, the two images may be of the same or substantially similar scenes but from slightly different angles. The two different images are combined in the user's brain, which attempts to make sense of the presented image information and, in this process, attaches depth information to the present images due to the slight differences between the two images.

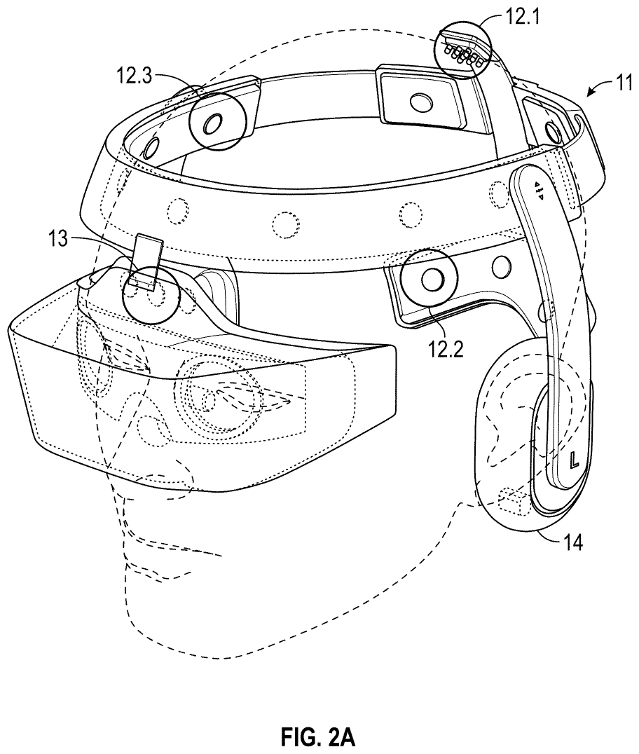

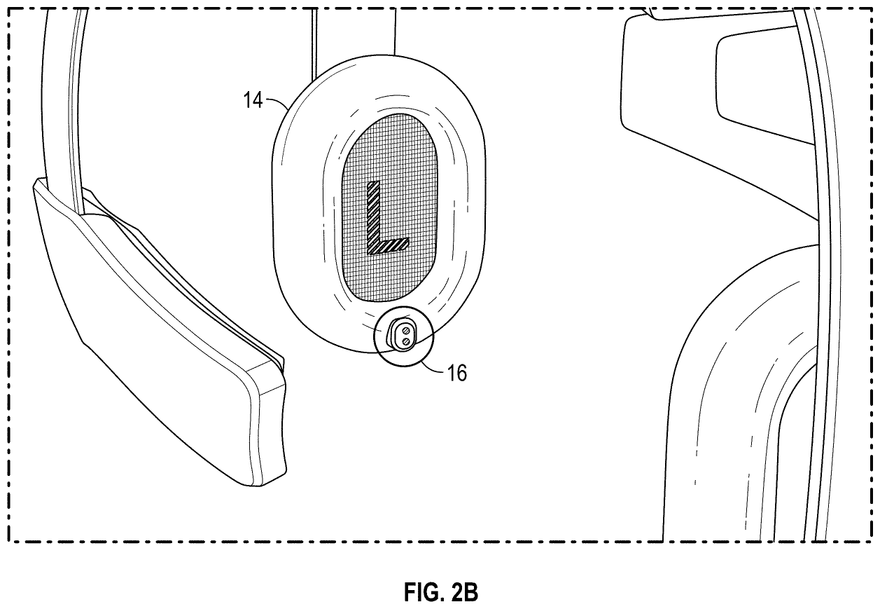

Referring to FIGS. 2A-2C, the virtual reality system may further include an electrode cap 11 and/or a controller 15. The electrode cap 11 may be a cloth cap (or hat) or the like that has a plurality of electrodes (e.g., EEG electrodes) 12.1, 12.2, and 12.3 embedded therein. The user 1 may wear the electrode cap 11 on his or her head. In some embodiments, the electrode cap 11 may be attached to the HMD 10, but the present disclosure is not limited thereto. For example, as shown in FIG. 2D, the electrode cap 11 may be separate from the HMD 10 such that the user 1 may decide to use the bioresponsive virtual reality system without the electrode cap 11 with a corresponding reduction in functionality, as will be understood based on the description below. In such an embodiment, the electrode cap 11 may be electrically connected to the HMD 10 by a connector (e.g., via a physical connection) or may be wirelessly connected to the HMD 10 by, for example, a Bluetooth® (a registered trademark of Bluetooth Sig, Inc., a Delaware corporation) connection or any other suitable wireless connection know to those skilled in the art. The electrode cap 11 may be embodied in a baseball hat to provide a pleasing aesthetic outward appearance by hiding the various electrodes 12.1, 12.2, and 12.3 in the electrode cap 11.

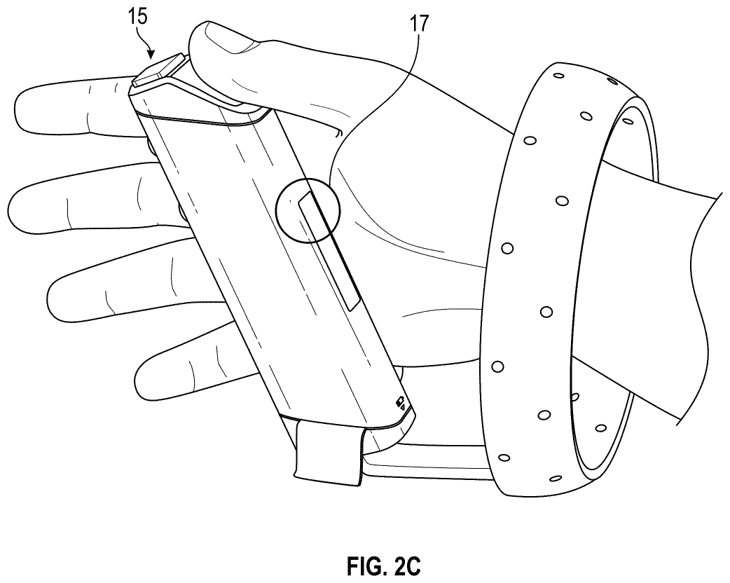

The electrodes 12.1, 12.2, and 12.3 in the electrode cap 11 may monitor the electrical activity of the brain of user 1. In some embodiments, the electrode cap 11 may be an electroencephalogram (EEG) cap. An EEG is a test that detects brain waves by monitoring the electrical activity of the brain of user 1. By monitoring brain wave activity at different areas of the brain of user 1, aspects of the emotional state of user 1 can be determined. FIG. 3 shows EEG results indicating different emotional states of the user 1.

The HMD 10 may also include headphones 14 for audio output, and heart rate sensors 16 arranged near the headphones 14. In some embodiments, the controller 15 may further monitor the heart rate of user 1. The heart rate sensor 14 may be an optical sensor configured to monitor the heart rate of user 1. The optical heart rate sensor may be, for example, a photoplethysmogram (PPG) sensor including a light-emitting diode (LED) and a light detector to measure changes in light reflected from the skin of user 1, which changes can be used to determine the heart rate of user 1.

The HMD 10 may also include blink detectors 13 configured to determine when the user 1 blinks.

The user 1 may interact with the displayed virtual reality environment by using the controller 15. For example, the controller 15 may include one or more gyroscopes (or accelerometers), buttons, etc. The gyroscopes and/or accelerometers in the controller 15 may be used to track the movement of the arm of user 1 (or arms when two controllers 15 are present). The controller 15 may be connected to the HMD 10 by a wireless connection, for example, a Bluetooth® connection. By using the output of the gyroscopes and/or accelerometers, the HMD 10 may project a virtual representation of the arm(s) of user 1 into the displayed virtual reality environment. Further, the user 1 may use the button on the controller 15 to interact with, for example, objects in the virtual reality environment.

The controller 15 may further include a galvanic skin response (GSR) sensor 17. In some embodiments, the controller 15 may be embodied as a glove, and the GSR sensor 17 may include a plurality of electrodes respectively contacting different ones of the fingers of user 1. By being embodied as a glove, the user 1 does not need to consciously attach the electrodes to his or her fingers but can instead put the glove on to place the electrodes in contact with the fingers. When the controller 15 is handheld, it may include two separate fingertip electrodes in recessed portions such that the user 1 naturally places his or her fingers on the two electrodes.

Galvanic skin response (GSR) (also referred to as electrodermal activity (EDA) and skin conductance (SC)) is the measurement of variations in the electrical characteristics of the skin of user 1, such as variations in conductance caused by sweating. It has been found that instances of increased skin conductance resulting from increased sweat gland activity may be the result of arousal of the autonomic nervous system.

The bioresponsive virtual reality system may further include other types of sensors, such as electrocardiogram (ECG or ECK) sensors and/or electromyography (EMG) sensors. The present disclosure is not limited to any particular combination of sensors, and it is contemplated that any suitable biophysiological sensor(s) may be included in the bioresponsive virtual reality system.

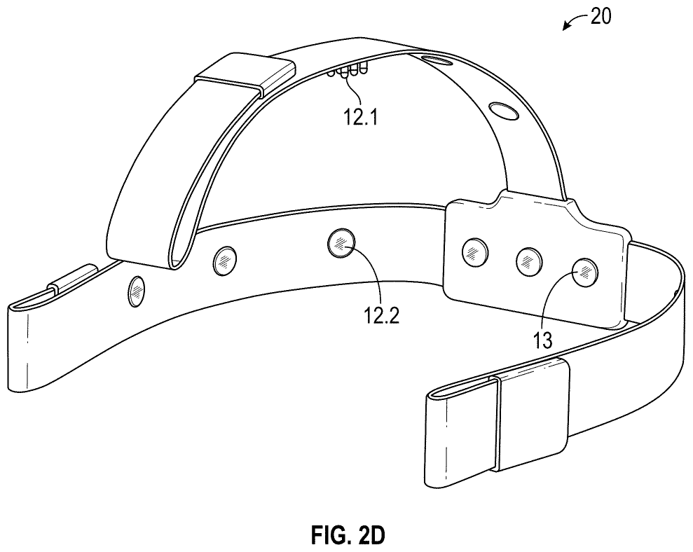

Referring to FIG. 4, the outputs (e.g., the measurements) of the EEG, GSR, and heart rate sensors (collectively, the “sensors”) may be input into a processor 30 of the bioresponsive virtual reality system. In some embodiments, as described above, the processor 30 may be integral with the display device, such as when a smartphone is used as a removal display device or, in other embodiments, may be separate from the display device and may be housed in the HMD 10.

The processor 30 may receive raw data output from the sensors and may process the raw data to provide meaningful information, or the sensors may process the raw data themselves and transmit meaningful information to the processor 30. That is, in some embodiments, some or all of the sensors may include their own processors, such as a digital signal processor (DSP), to process the received data and output meaningful information.

As will be further described below, the processor 30 receives the output of the sensors, calculates (e.g., measures and/or characterizes) the affective status of the user 1 based on the received sensor signals (e.g., determines the calculated affective state of user 1), and modifies the displayed content (e.g., the displayed virtual reality environment, the visual stimulus, and/or the displayed images) to put the user 1 into a target affective state or to maintain the user 1 in the target affective state. This method of modifying the displayed virtual reality environment based on biophysiological feedback from the user 1 may be referred to as bioresponsive virtual reality.

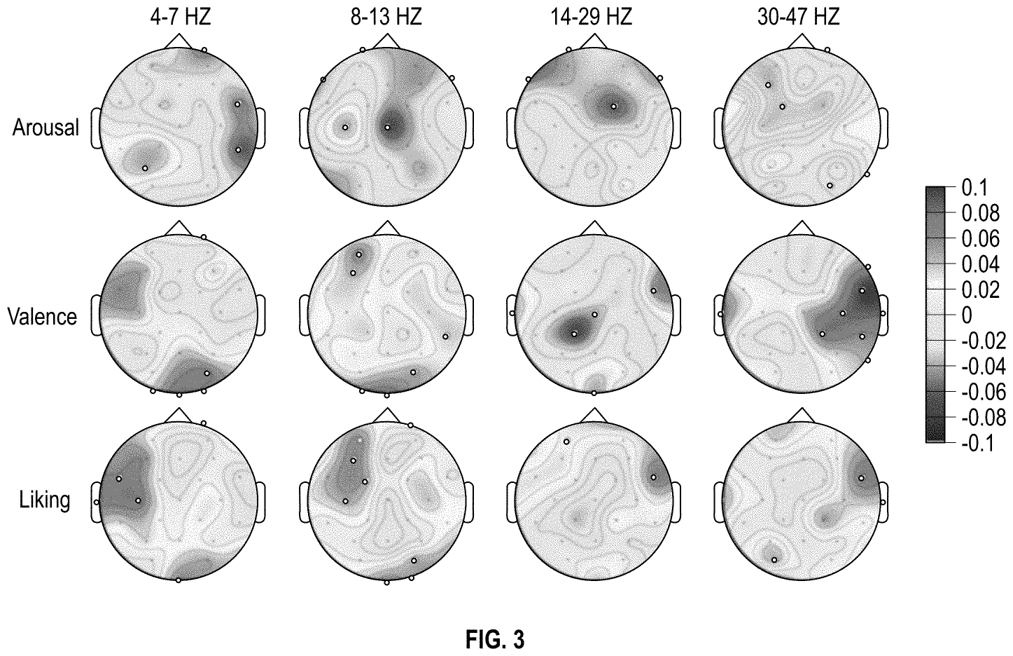

The bioresponsive virtual reality system may be applied to video games as well as wellbeing and medical applications as a few examples. For example, in a gaming environment, the number of enemies presented to the user 1 may be varied based on the calculated affective state of the user 1 as determined by the received sensor signals (e.g., the user's biophysiological feedback) to prevent the user 1 from feeling overly distressed (see, e.g., FIG. 5). As another example, in a wellbeing application, the brightness of the displayed virtual reality environment may be varied based on the calculated affective state of the user 1 to keep the user 1 in a calm or serene state (see, e.g., FIG. 5). However, the present disclosure is not limited to these examples, and it is contemplated that the displayed virtual reality environment may be suitably varied in different ways based on the calculated affective state of the user 1.

Referring to FIG. 5, different emotional (or affective) states are shown on a wheel graph. In modern psychology, emotions may be represented by two core affects—arousal and valence. Arousal may be a user's excitement level, and valence may be a user's positive or negative sense. By considering both arousal and valence, a user's affective state may be determined. Further, it has been found that EEG signals may be used to determine a user's valence, while GSR signals may be used to determine a user's arousal. Heart rate signals may be used to determine a user's emotional and/or cognitive states.

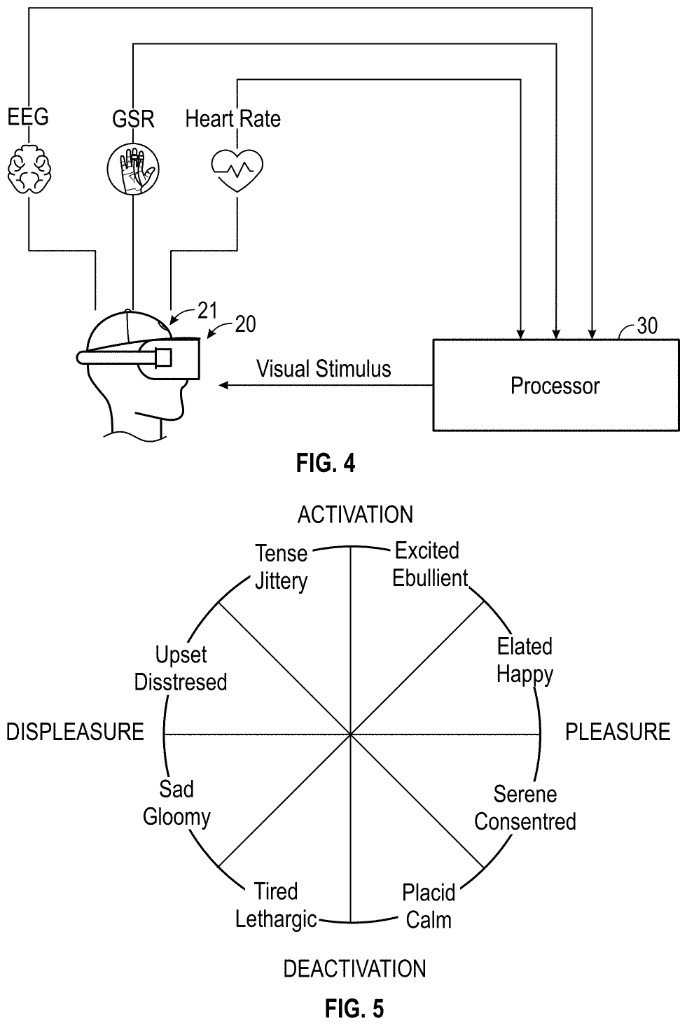

Referring to FIG. 6, an affective state classification network (e.g., affective state classification neural network) 50 is schematically illustrated. The affective state classification network 50 may be a part of the processor 30 of the virtual reality system (see, e.g., FIG. 4). The affective state classification network 50 may run on (e.g., the processor 30 may be or may include) a central processing unit (CPU), a graphics processing unit (GPU), and/or specialized machine-learning hardware, such as a TensorFlow Processing Unit (TPU)® (a registered trademark of Google Inc., a Delaware corporation), or the like.

The affective state classification network 50 may include a plurality of convolutional neural networks (CNNs) 52, one for each sensor input 51, and the CNNs 52 may output data to a neural network 53, such as a fully connected cascade (FCC) neural network, that calculate and outputs the user's affective state (e.g., the user's calculated affective state) 54 based on the output of the CNNs 52. The affective state classification network 50 may be a multi-modal deep neural network (DNN).

The affective state classification network 50 may be pre-trained on the general population. For example, the affective state classification network 50 may be loaded with a preliminary (or baseline) training template based on a general population of users. Training of the neural network(s) will be described in more detail below.

The CNNs 52 may receive the sensor inputs 51 and output a differential score based on the received sensor inputs 51 indicative of the user's arousal and valence states as indicated by each sensor input 51. For example, the CNN 52 corresponding to the GSR sensor input 51 may receive the output from the GSR sensor over a period of time and may then output a single differential value based on the received output 51 from the GSR sensor. For example, the CNN 52 may output a single numerical value indicative of the user's arousal level. Similarly, the CNN 52 corresponding to the EEG sensor input 51 may output a single numerical value indicative of the user's valence level.

The neural network 53 receives the numeral values from the CNNs 52, which are indicative of the user's arousal level and/or valence level, and outputs a single numerical value indicative of the user's affective state (e.g., the user's calculated affective state) 54. The neural network 53 may be preliminarily trained on the general population. That is, the neural network 53 may be loaded with a preliminary (or baseline) bias derived from training on a large number of members of the general population or a large number of expected users (e.g., members of the general population expected to use the bioresponsive virtual reality system). By pre-training the neural network 53 in this fashion, a reasonably close calculated affective state 54 may be output from the neural network 53 based on the different inputs from the CNNs 52.

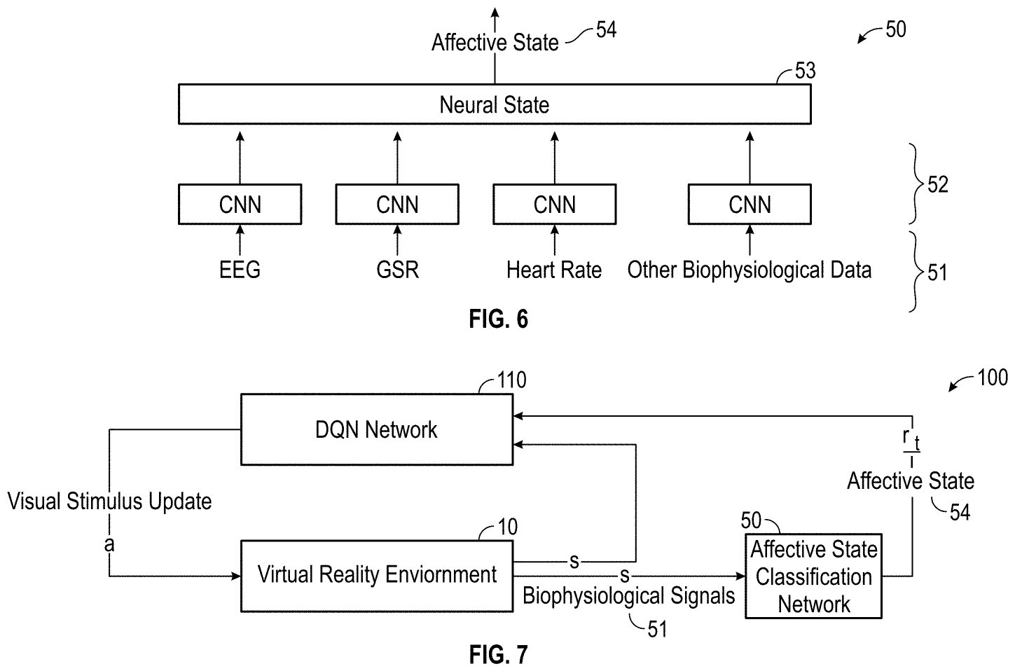

Referring to FIG. 7, a schematic diagram illustrating a control neural network (e.g., a closed-loop control neural network) 100 of the bioresponsive virtual reality system is shown. The control neural network 100 may be a part of the processor 30 (see, e.g., FIG. 4). For example, a Deep Q-Network (DQN) 110, further described below, may be a part of the processor 30 and may run on a conventional central processing unit (CPU), graphics processing unit (GPU), or may run on specialized machine-learning hardware, such as a TensorFlow Processing Unit (TPU)® or the like.

The control neural network 100 uses the DQN 110 to modify the virtual reality environment 10 (e.g., the modify visual stimulus of the virtual reality environment 10) displayed to the user via the HMD 10 based on the user's calculated affective state 54 as determined by the affective state classification network 50.

In the control neural network 100, the DQN 110 receives the output (e.g., the user's calculated affective state) 54 of the affective state classification network 50 and the currently-displayed virtual reality environment 10 (e.g., the virtual reality environment currently displayed on the HMD 10). The DQN 110 may utilize deep reinforcement learning to determine whether or not the visual stimulus being presented to the user in the form of the virtual reality environment 10 needs to be updated (or modified) to move the user into a target affective state or keep the user in the target affective state.

For example, a target affective state, which may be represented as a numerical value, may be inputted into the DQN 110 along with the currently-displayed virtual reality environment 10 and the user's current calculated affective state 54. The DQN 110 may compare the target affective state with the user's current calculated affective state 54 as determined by the affective state classification network 50. When the target affective state and the user's current calculated affective state 54 are different (or have a difference greater than a target value), the DQN 110 may determine that the visual stimulus needs to be updated to move the user into the target affective state. When the target affective state and the user's current calculated affective state 54 are the same (or have a difference less than or equal to a target value), the DQN 110 may determine that the visual stimulus does not need to be updated. In some embodiments, the DQN 110 may determine that the user's current calculated affective state 54 is moving away from the target affective state (e.g., a difference between the target affective state and the user's current calculated affective state 54 is increasing) and, in response, may update the visual stimulus before the target affective state and the user's current calculated affective state 54 have a difference greater than a target value to keep the user in the target affective state.

The DQN 110 may vary the visual stimulus changes (or updates) based on changes in the user's current calculated affective state 54. For example, the DQN 110 may increase the brightness of the virtual reality environment 10 in an attempt to keep the user within a target affective state. When the DQN 110 determines that the user's current calculated affective state 54 continues to move away from the target affective state after the changes in the brightness of the virtual reality environment 10, the DQN 110 may then return the brightness to the previous level and/or adjust another aspect of the virtual reality environment 10, such as the color saturation. This process may be continually repeated while the user is using the bioresponsive virtual reality system. Further, in some embodiments, the target affective state may change based on the virtual reality environment 10. For example, when the virtual reality environment is a movie, the target affective state input into the DQN 110 may change to correspond to different scenes of the movie. As one example, the target affective state may be changed to tense/jittery (see, e.g., FIG. 5) during a suspenseful scene, etc. In this way, the DQN 110 may continually vary the visual stimulus to keep the user in the target affective state, and the target affective state may vary over time, necessitating further changes in the visual stimulus.

The control neural network 100 may be trained to better correspond to a user's individual affective state responses to different content and/or visual stimulus. As a baseline model or value function (e.g., a pre-trained or preliminary model or value function), the affective state classification network 50 may be trained (e.g., pre-trained) on the general population. To train the control neural network 100 based on the general population, a set of content (e.g., visual stimulus), also referred to herein as “control content,” is displayed to a relatively large number of the general population while these users wear the bioresponsive virtual reality system. The sensor outputs 51 are input to the affective state classification network 50 (see, e.g., FIG. 6), which calculates an affective state for each person as he or she views the different control content. Then, the members of the general population will indicate their actual affective state while or after viewing each control content, and the actual affective state is used to train the affective state classification network 50 to more accurately calculate a calculated affective state 54 by correlating the sensor outputs 51 with actual affective states. As patterns begin to form in the data collected from the general population, the control content will be annotated (or tagged) with an estimated affective state. For example, when a first control content tends to evoke particular arousal and valance responses, the first control content will be annotated with those particular arousal and valance responses.

As one example, when the first control content is a fast-paced, hectic virtual reality environment, members of the general population may tend to feel tense/jittery when viewing the first control content. The members of the general population (or at least a majority of the general population) then report feeling tense/jittery when viewing the first control content, and the affective state classification network 50 would then correlate the sensor outputs 51 received while the members of the general population viewed the first control content with a tense/jittery affective state. However, it is unlikely that every member of the general population will have the same affective state response to the same virtual reality environment 10, so the affective state classification network 50 may determine patterns or trends in how the members of the general population respond to the first control content (as well as the other control content) to correlate the sensor outputs 51 with actual affective states as reported by the members of the general population and annotate the first control content accordingly.

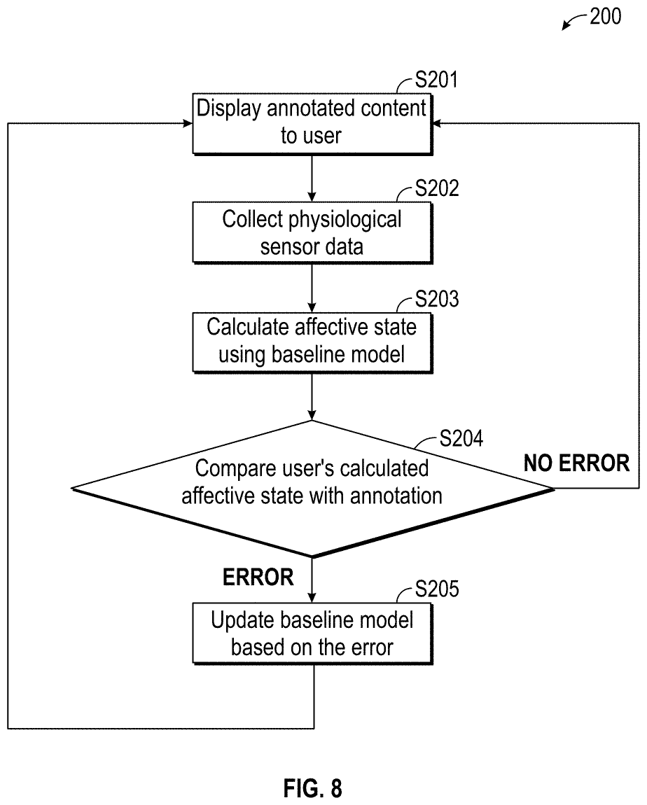

While the above-described method may provide a baseline model for the affective state classification network 50, it may not be accurate (e.g., entirely accurate) for a particular user (referred to as the “first user” herein) because one particular user may have different biophysiological responses to a virtual reality environment than an average member of the general public. Thus, referring to FIG. 8, a calibration process (e.g., a training process) 200 may be used to calibrate (or train) the affective state classification network 50 to the first user.

First, annotated content (e.g., annotated content scenes or annotated stimuli) is displayed to the first user via the HMD 10 (S201). The annotated content may be, as one example, control content that is annotated based on the results of the general population training or it may be annotated based on the expected affective state. While the first user is watching the annotated content on the HMD 10, the sensor outputs 51 from the biophysiological sensors, such as the EEG, GSR, and heart rate sensors, are received by the affective state classification network 50 (S202). The affective state classification network 50 then calculates the first user's affective state by using the baseline model (S203). The DQN 110 then compares the first user's calculated affective state with the annotations of the annotated content, which correspond to the expected affective state based on the general population training (S204). When the DQN 110 determines that an error exists between the first user's calculated affective state and the annotations of the annotated content, such as when the first user's calculated affective state does not match (or is not within a certain range of values of) the annotations of the annotated content, the DQN 110 will update the baseline model of the affective state classification network 50 to correlate the first user's detected biophysiological responses based on the sensor outputs 51 with the annotations of the annotated content (S205). And when the DQN 110 determines that an error does not exist between the first user's calculated affective state and the annotations of the annotated content, such as when the first user's calculated affective state matches (or is within a certain range of values of) the annotations of the annotated content, the DQN 110 will not make any changes to the affective state classification network 50.

The calibration process 200 continues by subsequently displaying annotated content to the first user until a number of the (e.g., all of the) annotated content has been displayed to the first user. For example, the calibration process 200 may be configured to run until all annotated content has been displayed to the first user.

After the affective state classification network 50 is calibrated to a particular user (e.g., the first user, as in the provided example above), the bioresponsive virtual reality system, such as the control neural network 100, will begin monitoring and calculating the user's affective state as the user views different content and will tailor (e.g., change or modify) the content viewed by the user such that the user achieves (or stays in) a target affective state, as discussed above.

Further, the DQN 110 may learn (e.g., may continuously learn) how changes to the visual stimulus affect the user's calculated affective state to make more accurate changes to the displayed visual stimulus. For example, the DQN 110 may execute a reinforcement learning algorithm (e.g., a value function), such as Equation 1, to achieve the target affective state.

Q^{π}(s, a)=E[rₜ₊₁+yrₜ₊₂+y²rₜ₊₃+ . . . |s, a] Equation 1:

wherein s is the user's calculated affective state output by the affective state classification network 50, rₜ is the reward (e.g., the target affective state), a is the action (e.g., the change in visual stimulus used to change the user's affective state), π is the policy that attempts to maximize the function (e.g., the mapping from the user's calculated affective state to the actions, such as to the changes in the visual stimulus), Q is the expected total reward (e.g., the user's expected resulting affective state), and y is a discount factor.

At each step, the value function, such as Equation 1, represents how good each action or state is. Thus, the value function provides the user's expected affective state based on the user's calculated affective state based on the sensor output 51 and the virtual reality environment 10 presented to the user based on the above-discussed trained policy with the discount factor.

The optimal value function (e.g., the maximum achievable value) is represented by Equation 2.

Q*(s, a)=max_{π}Q^{π}(s, a)=Q^{π*}(s, a) Equation 2:

The action to achieve the optimal value function is represented by Equation 3.

π*(s)=argmaxₐQ^{π*(}s, a) Equation 3:

In some embodiments, a stochastic gradient descent may be used to optimize the value function.

Accordingly, in one embodiment, the control neural network 100 uses a deep reinforcement learning model (e.g., a deep reinforcement machine learning model) in which a deep neural network (e.g., the DQN 110) represents and learns the model, policy, and value function.

Although the present disclosure has been described with reference to the example embodiments, those skilled in the art will recognize that various changes and modifications to the described embodiments may be made, all without departing from the spirit and scope of the present disclosure. Furthermore, those skilled in the various arts will recognize that the present disclosure described herein will suggest solutions to other tasks and adaptations for other applications. It is the applicant's intention to cover, by the claims herein, all such uses of the present disclosure, and those changes and modifications which could be made to the example embodiments of the present disclosure herein chosen for the purpose of disclosure, all without departing from the spirit and scope of the present disclosure. Thus, the example embodiments of the present disclosure should be considered in all respects as illustrative and not restrictive, with the spirit and scope of the present disclosure being indicated by the appended claims and their equivalents.

What is claimed is:

1. A bioresponsive virtual reality system comprising:
a head-mounted display comprising a display device, the head-mounted display being configured to display a three-dimensional virtual reality environment on the display device;
a plurality of bioresponsive sensors; and
a processor connected to the head-mounted display and the bioresponsive sensors, the processor being configured to:
receive signals indicative of a user's arousal and valance levels from the bioresponsive sensors;
calibrate a neural network to correlate the user's arousal and valance values to a calculated affective state;
calculate the user's affective state based on the signals; and
vary the virtual reality environment displayed on the head-mounted display in response to the user's calculated affective state to induce a target affective state.

2. The bioresponsive virtual reality system of claim 1, wherein the bioresponsive sensors comprise at least one of an electroencephalogram sensor, a galvanic skin response sensor, and/or a heart rate sensor.

3. The bioresponsive virtual reality system of claim 2, further comprising a controller.

4. The bioresponsive virtual reality system of claim 3, wherein the galvanic skin response sensor is a part of the controller.

5. The bioresponsive virtual reality system of claim 2, further comprising an electrode cap,
wherein electrode cap comprises the electroencephalogram sensor.

6. The bioresponsive virtual reality system of claim 2, wherein, to calibrate the neural network, the processor is configured to:
display content annotated with an expected affective state;
calculate the user's affective state based on the signals;
compare the user's calculated affective state with the annotation of the content; and
when the user's calculated affective state is different from the annotation of the content, modify the neural network to correlate the signals with the annotation of the content.

7. The bioresponsive virtual reality system of claim 2, wherein, to vary the virtual reality environment to achieve the target affective state, the processor is configured to use deep reinforcement learning to determine when to vary the virtual reality environment in response to the user's calculated affective state.

8. A bioresponsive virtual reality system comprising:
a processor and a memory connected to the processor;
a head-mounted display comprising a display device, the head-mounted display device being configured to present a three-dimensional virtual reality environment to a user; and
a plurality of bioresponsive sensors connected to the processor,
wherein the memory stores instructions that, when executed by the processor, cause the processor to:
receive signals from the bioresponsive sensors;
calibrate an affective state classification network;
calculate a user's affective state by using the affective state classification network; and
vary the virtual reality environment displayed to the user based on the user's calculated affective state.

9. The bioresponsive virtual reality system of claim 8, wherein the affective state classification network comprises a plurality of convolutional neural networks, one convolutional neural network for each of the bioresponsive signals, and a multi-modal network connecting these networks to each other.

10. The bioresponsive virtual reality system of claim 9, wherein the affective state classification network further comprises a fully connected cascade neural network,
wherein the convolutional neural networks is configured to output to the fully connected cascade neural network, and
wherein the fully connected cascade neural network is configured to calculate the user's calculated affective state based on the output of the convolutional neural networks.

11. The bioresponsive virtual reality system of claim 8, wherein, to calibrate the affective state classification network, the memory stores instructions that, when executed by the processor, cause the processor to:
input a baseline model that is based on the general population;
display annotated content to the user by using the head-mounted display, the annotation indicating an affective state relating to the annotated content;
compare the user's calculated affective state with the affective state of the annotation; and
when a difference between the user's calculated affective state and the affective state of the annotation is greater than a value, modify the baseline model to correlate the received signals with the affective state of the annotation.

12. The bioresponsive virtual reality system of claim 8, wherein, to vary the virtual reality environment, the memory stores instructions that, when executed by the processor, cause the processor to:
compare the user's calculated affective state with a target affective state; and
when a difference between the user's calculated affective state and the target affective state is greater than a value, vary the virtual reality environment to move the user toward the target affective state.

13. The bioresponsive virtual reality system of claim 12, wherein, to vary the virtual reality environment, the memory stores instructions that, when executed by the processor, cause the processor to use a deep reinforcement learning method to correlate variations of the virtual reality environment with changes in the user's calculated affective state.

14. The bioresponsive virtual reality system of claim 13, wherein the deep reinforcement learning method uses Equation 1 as the value function, Equation 1:
Q^{π}(s, a)=E[rₜ₊₁+yrₜ₊₂+y²rₜ₊₃+ . . . |s, a]
wherein:
s is the user's calculated affective state;
rₜ is the target affective state;
a is the varying of the virtual reality environment;
π is the mapping of the user's calculated affective state to the varying of the virtual reality environment;
Q is the user's expected resulting affective state; and
y is a discount factor.

15. A method of operating a bioresponsive virtual reality system, the method comprising:
calibrating an affective state classification network;
calculating a user's affective state by using the calibrated affective state classification network; and
varying a three-dimensional virtual reality environment displayed to the user when the user's calculated affective state is different from a target affective state.

16. The method of claim 15, wherein the calculating the user's affective state comprises:
receiving signals from a plurality of biophysiological sensors;
inputting the received signals into a plurality of convolutional neural networks, the convolutional neural networks being configured to classify the signals as indicative of the user's arousal and/or valance levels; and
inputting the user's arousal and/or valance levels into a neural network, the neural network being configured to calculate the user's affective state based on the user's arousal and/or valance levels.

17. The method of claim 16, wherein the biophysiological sensors comprise at least one of an electroencephalogram sensor, a galvanic skin response sensor, and/or a heart rate sensor.

18. The method of claim 17, wherein the calibrating of the affective state classification network comprises:
displaying a three-dimensional virtual reality environment having an annotation to the user, the annotation indicating an affective state relating to the virtual reality environment;
comparing the user's calculated affective state with the affective state of the annotation; and
when a difference between the user's calculated affective state and the affective state of the annotation is greater than a threshold value, modifying the affective state classification network to correlate the received biophysiological signals with the affective state of the annotation.

19. The method of claim 15, wherein the varying of the three-dimensional virtual reality environment comprising:
receiving the target affective state;
comparing the user's calculated affective state with the target affective state;
varying the three-dimensional virtual reality environment when a difference between the user's calculated affective state and the target affective state is greater than a threshold value;
recalculating the user's affective state after the varying of the three-dimensional virtual reality environment; and
comparing the user's recalculated affective state with the target affective state.

20. The method of claim 19, wherein a deep-Q neural network is used to compare the user's calculated affective state with the target affective state.
